SEQUENCE LISTING (10 sequences)

SEQ ID NO: 1 - DNA, 40 bp, Artificial Sequence
  PCR amplification primer
cggcagatctctggatccatggagatgggcagacggattc

SEQ ID NO: 2 - DNA, 31 bp, Artificial Sequence
  PCR amplification primer
cgccgtcgacttagtcatcatcttctccctc

SEQ ID NO: 3 - DNA, 28 bp, Artificial Sequence
  PCR amplification primer
ccggcatatgtcagacgcagccgtagac

SEQ ID NO: 4 - DNA, 31 bp, Artificial Sequence
  PCR amplification primer
ccggctcgaggtcatcctcgtcggtcttctg

SEQ ID NO: 5 - RNA, 19 nt, Artificial Sequence
  siRNA
ucaccaccaaggacuuaaa

SEQ ID NO: 6 - RNA, 20 nt, Artificial Sequence
  siRNA
gcagcacgacuucuucaagu

SEQ ID NO: 7 - DNA, 20 bp, Artificial Sequence
  RT-PCR primer
atgatctcggatgaccaaac

SEQ ID NO: 8 - DNA, 20 bp, Artificial Sequence
  RT-PCR primer
ggaggcggctgcggcgagca

SEQ ID NO: 9 - DNA, 20 bp, Artificial Sequence
  RT-PCR primer
tccaccaccctgttgctgta

SEQ ID NO: 10 - DNA, 20 bp, Artificial Sequence
  RT-PCR primer
accacagtccatgccatcac

(19) United States
(12) Patent Application Publication
(10) Pub. No.: US 2005/0080102 A1
(43) Pub. Date: Apr. 14, 2005

(54) Compound and uses thereof

(76) Inventors: Saul Rosenberg, Grayslake, IL (US); Shi Chung Ng, Libertyville, IL (US); James Kofron, Bristol, WI (US); Haichao Zhang, Lake Bluff, IL (US); Xuejun Jiang, New York, NY (US); Xiaodong Wang, Dallas, TX (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/889,629

(22) Filed: Jul. 12, 2004

Related U.S. Application Data
(63) Continuation of application No. 10/659,850, filed on Sep. 11, 2003, now abandoned.
(60) Provisional application No. 60/410,238, filed on Sep. 12, 2002.

(51) Int. Cl. A61K 31/44; C07D 213/28; G01N 33/574

(52) U.S. Cl. 514/277; 546/339; 435/7.23

(57) ABSTRACT

The subject invention relates to a novel small molecule, referred to as alpha-(trichloromethyl)-4-Pyridineethanol (PETCM), as well as to uses thereof. PETCM was identified and isolated by high throughput screening and is an activator of caspase-3. Using PETCM in combination with biochemical fractionation, a novel pathway that regulates mitochondria-initiated caspase activation was also identified. This pathway comprises tumor suppressor PHAP proteins and oncoprotein prothymosin-alpha. PETCM relieves prothymosin-alpha inhibition and allows apoptosome to form at a physiological concentration of dATP.

The present application claims priority to U.S. Provisional Application No. 60/410,238, filed on Sep. 12, 2002, hereby incorporated in its entirety by reference.

### BACKGROUND OF THE INVENTION

1. Technical Field

The subject invention relates to a novel small molecule, referred to as alpha-(trichloromethyl)-4-Pyridineethanol (PETCM), as well as to uses thereof. PETCM was identified and isolated by high throughput screening as a compound that enhances caspase-3 activation in a cell-extract system. Caspase-3 is a downstream effector of apoptosis and is responsible for the cleavage of multiple cellular substrates in the cell death process (Hengartner, M. O., The biochemistry of apoptosis [Review], Nature, 2000, 407, 770-776)). Such substrates include PARP, ICAD, cytoskeletal proteins and other proteins essential for survival. Hence, caspase 3 is regarded as the terminal caspase in the cascade of caspase activation. Using PETCM in combination with biochemical fractionation, a novel pathway that regulates mitochondria-initiated caspase activation was also identified. This pathway comprises tumor suppressor PHAP proteins and oncoprotein prothymosin-alpha. PETCM relieves prothymosin-alpha inhibition and allows apoptosome to form at a physiological concentration of dATP.

2. Background Information

Holocytochrome c release from mitochondria to cytosol marks a defined moment in mammalian cells' response to a variety of apoptotic stimuli. The rapidness and thoroughness of the release disrupt the normal electron transfer chain and activate apoptotic caspases (Goldstein et al., Natl. Cell Biol. 2:15 (2002); Wang et al., Genes Dev. 15:2922 (2001)). The released cytochrome c readily binds to Apaf-1 and induces a conformational change that allows stable binding of dATP/ATP to Apaf-1, an event that drives the formation of an heptamer Apaf-1/cytochrome c complex named apoptosome (Jiang et al., J. Biol. Chem. 275:31199 (2000); Acehan et al., Mol. Cell. 9:423 (2002)). Apoptosome recruits procaspase-9 and induces auto-activation thereof. The apoptosome-bound caspase-9 cleaves and activates the downstream caspases such as caspase-3, -6, and -7 (Li et al., Cell 91:479 (1997); Rodriguez et al., Genes Dev. 13:1379-(1999)). These caspases then cleave many intracellular substrates leading to the characteristic apoptotic death and phagocytosis of the dead cells (Thornberry et al., Science 2181:1312 (1998)).

The mitochondrial caspase activation pathway is closely regulated. One major regulatory step is at the release of cytochrome c from mitochondria, a process controlled by the Bcl-2 family of proteins, which includes both pro-death and anti-death members (Adams et al., Science 281:1312 (1998); Chao et al., Annu. Rev. Immunol. 16:395 (1998)). On the other hand, the IAP proteins regulate the pathway by directly inhibiting caspase activity (Wang, Genes Dev. 15:2922 (2001); Deveraux et al., Genes Dev. 13:239 (1999)). The inhibitory activity of IAP can be antagonized by mitochondrial proteins such as Smac/Diablo and Omi/HtrA2 after they are released to cytoplasm (Du et al., Cell 102:33 (2000); Verhagen et al., Cell 102:43 (2000); Verhagen et al., Cell 102:445 (2001); Suzuki et al., Mol. Cell 8:613 (2001); Hegde et al., J. Biol. Chem. 277:432 (2001)).

In view of the above, there is a need for a thorough understanding of the caspase activation pathway as well as particular activators thereof. The present invention provides such an understanding as well as the isolation and identification of such an activator.

### SUMMARY OF THE INVENTION

The subject invention relates to an activator of caspase-3 (i.e., PETCM) identified by use of high throughput screening, as well as to uses thereof. This compound plays a role in a novel death regulatory pathway that comprises tumor suppressor PHAP proteins and oncoprotein prothymosin-alpha, which play distinctive roles in regulating apoptosome formation and activity.

More specifically, the present invention encompasses a compound comprising alpha-(trichloromethyl-4-Pyridineethanol (PETCM) and as well as derivatives thereof. The compound may itself be alpha-(trichloromethyl-4-Pyridineethanol (PETCM) and may be isolated by high throughput screening (HTS).

The present invention also includes a method of activating a caspase pathway (e.g., the caspace-3 pathway) in a cell comprising the step of exposing PETCM to the cell in an amount sufficient to effect the activation. The cell may be mammalian and may be malignant. Such a malignant cell may be, for example, a colon cancer cell, a prostate cancer cell, a leukemia cell, a melanoma cell, a lymphoma cell, a cervical carcinoma or a glioblastoma cell. The PETCM may be exposed to the cell in a dosage in the range of approximately 0.1 uM to 1.0 mM. Preferably, a concentration of 0.2 mM is utilized.

Additionally, the present invention encompasses a method of inducing apoptosome formation in a cell, wherein the formation is inhibited by ProT, comprising the step of exposing PETCM to the cell in an amount sufficient to effect the induction.

The present invention also includes a method of inducing function of PHAP protein, in a cell, inhibited by prothymosin-alpha (ProT) comprising the step of exposing PETCM to the cell in an amount sufficient to induce the function.

Additionally, the present invention includes a method of reversing inhibition of caspace-3 activation, in a cell, wherein the inhibition is induced by ProT, comprising the step of exposing PETCM to the cell in an amount sufficient to effect the reversal.

Furthermore, the present invention includes a method of negatively regulating caspase-9 activation in a cell comprising the step of exposing ProT to the cell in an amount sufficient to negatively regulate activation thereof.

Moreover, the invention also includes a method of promoting caspase activation in a cell, subsequent to apoptosome formation, comprising administering PHAP protein to the cell in an amount sufficient to effect caspase activation.

The present invention also encompasses a method of isolating and identifying at least one protein which inhibits or activates an apoptopic pathway. This method comprises the steps of preparing fractions of a cellular extract; exposing the fractions to PETCM and determining whether apoptosis activation or inhibition occurs in each of the fractions; purifying the fractions which exhibit apoptosis activation or inhibition upon exposure to PETCM; and isolating from the purified fractions at least one protein, wherein the at least one protein inhibits or activates apoptosis in the apoptopic pathway. The at least one protein which activates apoptosis may be, for example, PHAPI, PHAP12a or PHAPIII. The at least one protein which inhibits apoptosis may be, for example, promothymosin-alpha.

Additionally, the present invention includes a method of identifying regulators of apoptosome formation. This method comprises the steps of preparing extracts of mammalian, malignant cells; exposing the extracts to a probe, wherein the probe comprises a nucleotide sequence encoding prothymosin-alpha, for a time and under conditions sufficient for complexes to form between the probe and nucleic acid sequences in the extracts; and detecting complex formation between the probe and the nucleic acid sequences in the extracts, wherein the nucleic acid sequences encode regulators of apoptosome formation.

### BRIEF DESCRIPTION OF THE DRAWINGS

FIG. 1 illustrates that PETCM stimulates caspase-3 activation and drives apoptosome formation in HeLa cell cytosol. More specifically, FIG. 1(A) illustrates the structure of PETCM. FIG. 1(B) illustrates that PETCM stimulates DEVD activity of HeLa S-100 in a dose-dependent manner. FIG. 1(C) represents a time course comparison of the stimulatory effects of PETCM and dATP. FIG. 1(D) illustrates that PETCM drives apoptosome formation.

FIG. 2 illustrates that stimulatory activity may be used to mediate the PETCM effect. FIG. 2(A) shows the fractionation scheme used. FIG. 2(B) illustrates the reconstitution of PETCM response with the fractions. Procaspase-3 (PC3) and the cleaved products are marked by arrows.

FIG. 3 illustrates the purification of the stimulatory activity or factor in Q100. FIG. 3(A) illustrates the purification scheme. FIG. 3(B) illustrates the activity assay of the fractions from the final Mono Q chromatography. FIG. 3(C) illustrates resolution of the final Mono Q fractions (30-μl each) and presence of the three purified proteins, PHAPI, PHAPI2a, and PHAPIII. FIG. 3(D) illustrates protein sequence alignment of PHAP proteins. The leucine-rich repeat cap (corresponding to residue 128-146 of PHAPI) is line-marked.

FIG. 4 illustrates purification of the inhibitory activity in Q100. FIG. 4(A) illustrates caspase-3 activation of various mixtures. FIG. 4(B) illustrates the purification scheme of the inhibitory activity. FIG. 4(C) illustrates the activity assay of the fractions from the final Mono Q chromatography. Caspase-3 activation of each mixture was measured. FIG. 4(D) illustrates resolution of the final Mono Q fractions.

FIG. 5 illustrates regulation of apoptosome by ProT and PHAP. FIG. 5(A) illustrates that PHAP accelerates caspase-3 activation after PETCM antagonizes the inhibitory activity of ProT. FIG. 5(B) illustrates that ProT inhibits apoptosome formation, and PETCM can antagonize the inhibitory activity. FIG. 5(C) illustrates that PHAP enhances caspase-9 activation. Apoptosome formation was measured as described in FIG. 1.

FIG. 6 shows the elimination of ProT by RNAi sensitized UV-induced apoptosis in HeLa cells. FIG. 6(A) illustrates RT-PCR, showing disruption of ProT messenger RNA by RNAi. FIG. 6(B) illustrates that ProT RNAi sensitizes UV-induced cell death. Cells were treated with ProT or GFP RNAi. Top panel shows microscopic pictures without UV treatment or 12 hr after UV irradiation. Bottom panel shows cell death counting using Hoechst staining at indicated time after UV irradiation. FIG. 6(C) illustrates that ProT RNAi increases UV-induced caspase-3 activation. FIG. 6(D) illustrates that the disruption of ProT by RNAi negates the PETCM requirement for caspase-3 activation.

### DETAILED DESCRIPTION OF THE INVENTION

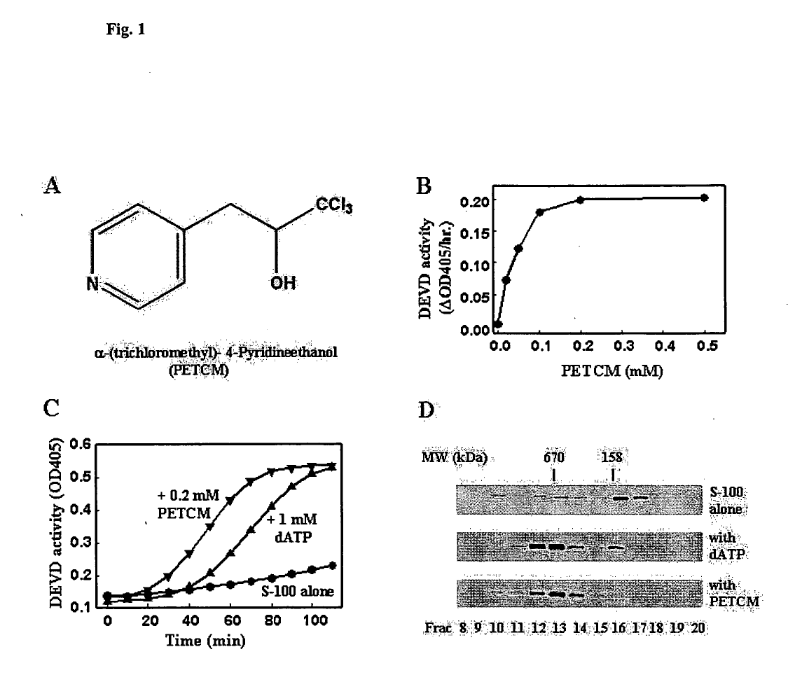

In an attempt to screen for small molecules that activate caspases, 184,000 compounds were screened for caspase-3 activator activity using HeLa cell extracts (see Example I). The most potent, positive hits from this large-scale, high throughput screening effort turned out to be from the novel compound alpha-(trichloromethyl)-4-Pyridineethanol, PETCM (FIG. 1A). This molecule has a simple chemical structure and has no chemical resemblance to dATP. The present invention encompasses this molecule, derivatives thereof, as well as methods of using this novel molecule.

As shown in FIGS. 1B and 1C, increasing amounts of PETCM added to cells result in significant caspase-3 activation as measured, for example, by the liberation of colorimetric artificial caspase-3 substrate Ac-DEVD-pNA (Bachem L1945). Other means of measuring caspoase-3 activation are also encompassed herein and are readily known to those of ordinary skill in the art.

The effective concentration for caspase-3 activation is between 0.1 uM to 1.0 mM. In particular, at 0.2 mM, PETCM was more efficient in activating caspase-3 than 1.0 mM dATP. Thus, the present invention encompasses a method of activating caspase-3, in cells, by administering this dosage (i.e., approximately at least 0.1 uM to 1.0 mM) of PETCM to the cells or exposing the cells to this dosage.

In order to find out how this small molecule (i.e., PETCM) promotes activation of caspase-3, apoptosome formation was analyzed using gel-filtration chromatography followed by Western blot analysis against Apaf-1 (Zou H et al., Cell 90:405-413, 1997). Other methods known to those of ordinary skill in the art may also be used for such an analysis. As shown in FIG. 1D, Apaf-1 in the normal HeLa cell S-100 was mostly in its inactive monomeric form. After incubating with 1 mM dATP, most of the Apaf-1 was shifted to the size of ˜1 million Dalton, indicating formation of apoptosome. After incubating S-100 with 0.2 mM of PETCM, Apaf-1 was also shifted to the position of apoptosome. The efficiency of apoptosome formation is better than 1 mM dATP, a result that was consistent with the caspase-3 assay (FIG. 1C).

Extracts from a battery of human tumor cells were also screened for their response to PETCM. It was determined that many human cancer lines, including colon cancer, prostate cancer, promyelocytic leukemia, T cell leukemia, bone marrow leukemia, malignant melanoma, lymphoma, and glioblastoma cells were responsive. Cervical carcinoma cells as well as other carcinoma cells with functional prothymosin alpha inhibitory pathways may also e responsive to PETCM. PETCM and the PETCM-stimulated caspase activation pathway are therefore of fundamental and clinical significance with respect to malignant mammalian cells. Thus, the present invention encompasses a method of stimulating caspase activity in malignant cells comprising exposing said cells to PETCM or administering PETCM to said cells.

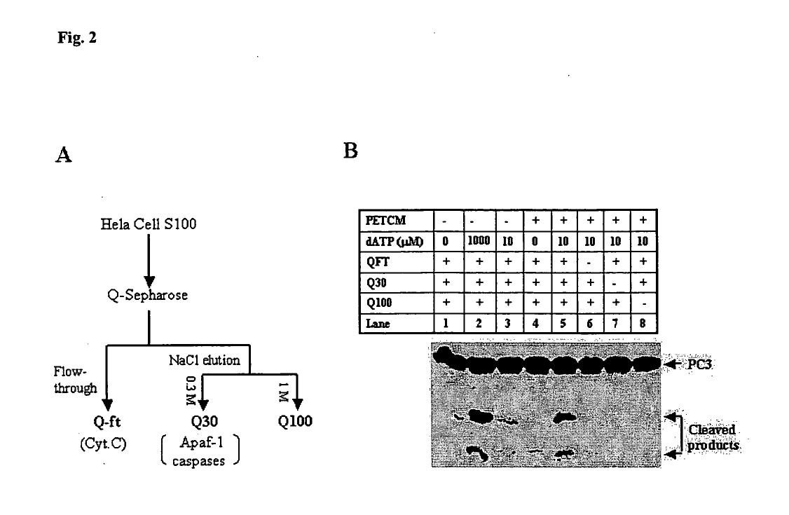

Further, it was not clear from previous knowledge on cellular apoptotic pathways, and PETCM chemical structure, how PETCM actually promotes apoptosome formation and caspase-3 activation. To study the mechanism, HeLa cell S-100 extracts were fractionated using an anion exchange column. As shown in FIG. 2A, three fractions were prepared. The first fraction, Q-ft, flew through the column and contained cytochrome c (Liu et al., Cell 86:147 (1996)). The second fraction, Q30, was eluted with 0.3 M NaCl and contained Apaf-1 (Zou et al., Cell 90:405 (1997)) and procaspase-9 (Li et al., Cell 91:479 (1997)). The third fraction, Q100, was eluted with 1 M NaCl. These three fractions were then used in different combinations to search for proteins that might mediate the PETCM effect.

As shown in FIG. 2B, when all three fractions were incubated together in the presence of 10 μM dATP (i.e., the physiological concentration in cells), little caspase-3 activation was observed (lane 3). In contrast, when 1 mM dATP was used, robust caspase-3 activation (lane 2) was observed. However, in the presence of 0.2 mM PETCM, caspase-3 activation was observed at 10 μM dATP, indicating that the combination of these three fractions mimicked what happened in S-100 (lane 5). No caspase-3 activation was seen if dATP was completely omitted, indicating that PETCM function still requires dATP (lane 4). Omitting Q-ft (cytochrome c), or Q30 (Apaf-1/procaspase-9) diminished caspase-3 activating activity (lanes 6-7). Surprisingly, omitting Q100 also significantly reduced caspase-3 activating activity (lane 8). This experiment suggested that Q100 contained unknown protein factor(s) that mediated the stimulating effect of PETCM.

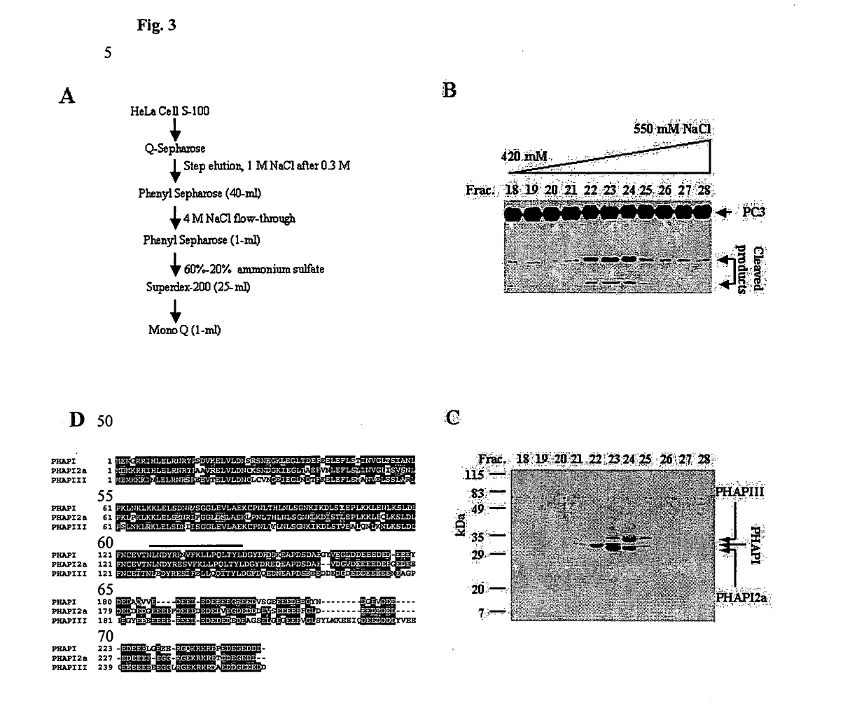

The stimulatory activity in Q100 was purified by chromatography (FIG. 3). The result of the final Mono Q column was shown in FIG. 3B. A single activity peak at fractions 22-24 was observed. When these fractions were subjected to SDS-PAGE followed by silver staining, three proteins with molecular weights of 35, 32, and 29 kDa showed perfect correlation with the activity (FIG. 3C).

These three proteins were identified by mass spectrum analysis as PHAPI (also called PP32 and LANP) (Vaesen et al., Biol. Chem. Hoppe-Seyler 375:113 (1994); Chen et al., Mol. Biol. Cell 7:2045 (1996); Matilla et al., Nature 389:974 (1997)), PHAPI2a (also called SSP29 and April) (Zhu et al., Biocehm. Mol. Biol. Int. 42:927 (1997); Mencinger et al., Biochim. Biophys. Acta. 1395:176 (1998)), and a theoretical protein in the NCBI database, which was termed PHAPIII. The three proteins are closely related and share over 80% identical amino acid sequence (FIG. 3D). They have a long acidic C-terminus and a leucine-rich region in the middle (FIG. 3D). In mammalian cells, PHAP proteins are putative tumor suppressors (Chen et al., Mol. Biol. Cell 7:2045 (1996); Brody et al., J. Biol. Chem. 274:20053 (1999); Bai et al., Oncogene 20:2153 (2001)), a function consistent with the pro-apoptotic activity identified here.

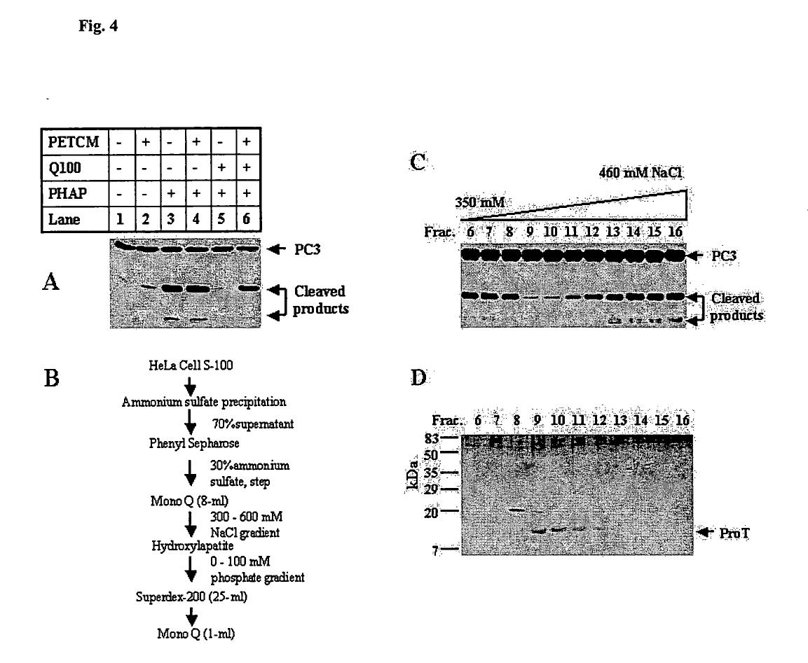

After identification of PHAP proteins, confirmation of their caspase stimulatory activity and dependence on PETCM was carried out. Surprisingly, when purified PHAP proteins were used to stimulate caspase-3 activation, the stimulatory effect of PHAP proteins was independent of PETCM (FIG. 4A, lane 1-4). However, if Q100 was added back, from which PHAP was purified, the stimulatory activity of PHAP was suppressed and the suppression was reversed by the addition of PETCM (lanes 5-6). This finding indicated that there was an inhibitory factor in the Q100 fraction as well. The PHAP proteins could only function when the inhibitory factor was antagonized by PETCM.

A strategy was derived to purify this inhibitory factor from HeLa cell S-100. The inhibitory activity was assayed by adding column fractions to the mixture of Q30/cytochrome c/PHAP/dATP. A single inhibitory factor was purified using a six-step chromatography procedure (FIG. 4B). FIG. 4C and FIG. 4D show the activity and the silver stained gel of the final Mono Q column. The protein was identified by mass spectrum analysis as prothymosin-alpha (ProT) (Dosil et al., J. Biol. Chem. 276:1794 (2001)).

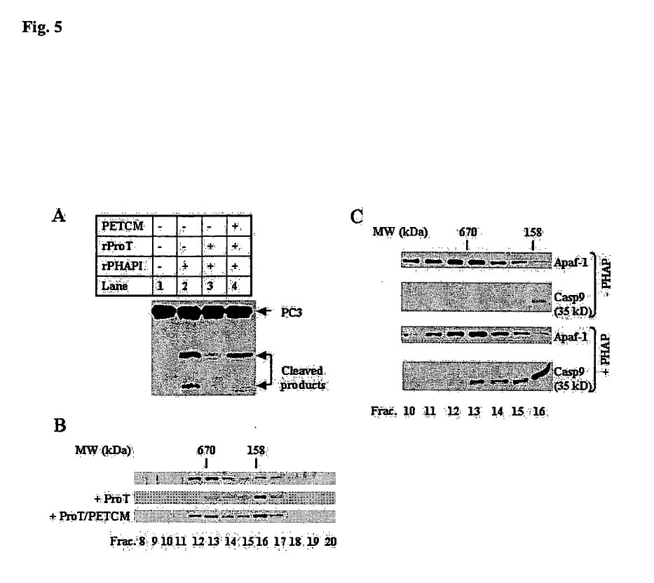

FIG. 5A demonstrates the final reconstitution of the PETCM initiated regulatory pathway. Recombinant PHAPI stimulated caspase-3 activation when added to the Q30 fraction plus cytochrome c and 10 μM dATP (lane 2). The activity was inhibited when recombinant ProT was included in the reaction (lane 3), and the inhibitory effect of ProT was reversed in the presence of PETCM (lane 4). Subsequently, regulation of apoptosome by these players was tested. As shown in FIG. 5B, in the presence of ProT, formation of apoptosome was efficiently blocked and PETCM relieved the blockage when present in the reaction. In contrast, the presence of PHAPI did not affect the efficiency of apoptosome formation. Instead, more activated caspase-9 was observed and there was also more caspase-9 associated with apoptosome (FIG. 5C). Pull-down experiments also showed more association of active caspase-9 with Apaf-1 in the presence of PHAPI. These results indicate that ProT and PHAP regulate caspase-3 activation at different steps. ProT inhibits caspase-3 activation by blocking apoptosome formation and therefore acts more upstream in this regulatory pathway, while PHAPI does not affect apoptosome formation but accelerates its activity to promote more caspase-9 activation. PETCM promotes caspase-3 activation by removing the inhibition of ProT on apoptosome formation, allowing PHAPs to stimulate apoptosome activity.

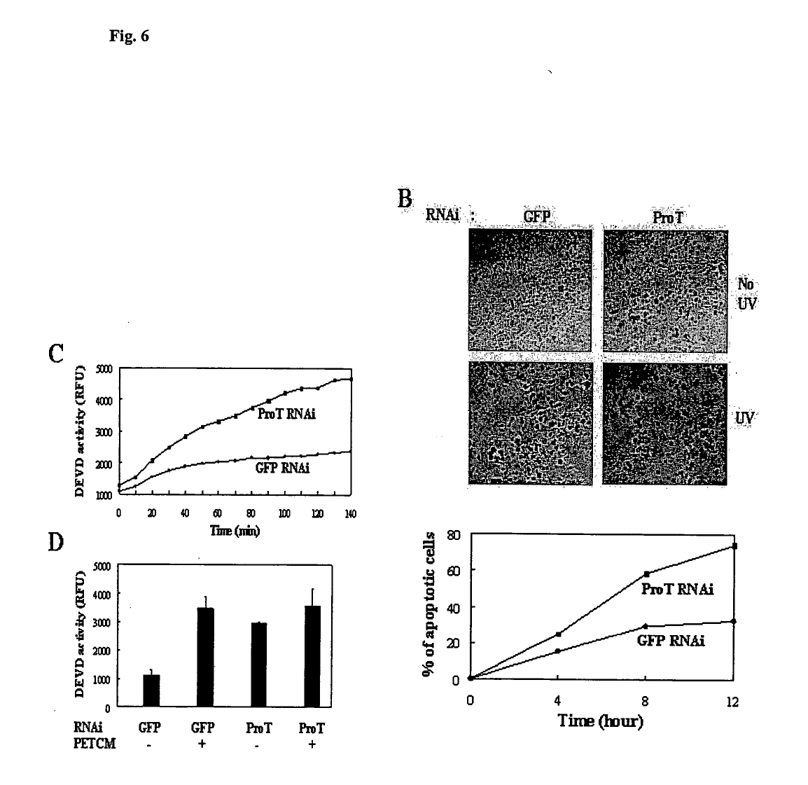

To verify the apoptotic roles of PHAP and ProT in vivo, an attempt was made to eliminate their expression from HeLa cells by RNA interference (RNAi). RNAi against PHAP proteins did not work, probably because there are multiple forms of PHAP and they are stable proteins. On the other hand, RNAi against ProT worked well. As shown in FIG. 6A, RNAi against ProT efficiently eliminated the ProT mRNA. Under this condition, the cells are alive and no obvious apoptosis was observed. However, when irradiated with UV light, the cells treated with ProT RNAi showed a much higher rate of apoptosis as shown in FIG. 6B. Thus, 12 hours after UV irradiation, more than 70% of the ProT RNAi treated cells showed apoptotic morphology while a control RNAi (GFP) treated cells only showed 25% cell death. The cell death was correlated with the caspase-3 activation since higher caspase-3 activity was also observed in the ProT RNAi treated cells (FIG. 6C).

The RNAi experiment also confirmed that PETCM functioned to antagonize the inhibitory activity of ProT. As shown in FIG. 6D, the extracts from control RNAi treated-cells were responsive to PETCM. In contrast, cell extracts from ProT RNAi treated cells were able to activate caspase-3 independent of PETCM.

The inhibition of apoptosome formation by ProT offered an explanation for a long-standing puzzling observation that up to millimolar level of dATP is required to trigger efficient caspase-3 activation in vitro.

The results presented herein indicate that cells must have ways to antagonize ProT during apoptosis, an effect that is “hijacked” by PETCM (i.e., mimics action of an endogenous, but unidentified antagonist of ProT), and the release of cytochrome c from mitochondria alone may not always be sufficient to trigger apoptosis. This is consistent with the observation that microinjection of cytochrome c to healthy neurons did not induce apoptosis unless the cells first enter the stage of ‘competent to die’, which can be caused by NGF withdrawal (Deshmukh et al., Neuron 21:695 (1998)).

The finding that PETCM functions through ProT should also point to ways to study the intracellular pathways that regulate ProT activity.

In view of the above, the present invention relates to a small molecule referred to as PETCM, derivatives thereof, as well as methods of using the molecule in connection with the caspase activation pathway. The effectiveness of PETCM in a panel of cancer cells indicates the potential clinical value of the chemical and the pathway. Furthermore, PETCM may also be used in the discovery of other proteins or biomolecules involved in the apoptotic pathway.

The present invention may be illustrated by the use of the following non-limiting examples:

### EXAMPLE I

### Identification of Caspase-3 Activator Using High Throughput Screening

With respect to Example I and those which follow, nucleotide dATP was purchased from Pharmacia (Piscataway, N.J.). Horse heart cytochrome C(C7752) was purchased from Sigma (St. Louis, Mo.). Colormetric and fluorogenic caspase-3 peptide substrates were from CalBiochem (La Jolla, Calif.). Polyclonal anti-Apaf-1 antibody was prepared as described previously (Zou, et al., J. Biol. Chem. 274, 11549 (1999)). Anti-caspase-9 antibody (#9505) was purchased from Cell Signaling (Beverly, Mass.). All of the cell lines were purchased from the American Type Culture Collection, Manassas, Va. Protein concentration was determined by the Bradford method. General biochemical and molecular biology methods were performed as described in Molecular Cloning (Sambrook et al., 1989).

With respect to Example I, the high throughput screening (HTS) was essentially a cell-lysate assay in which the endpoint, activation of caspase-3, was monitored by the cleavage of a calorimetric substrate. HeLa cell lysate was prepared by Cellex Bioscience (Minneapolis, Minn.). This lysate was thawed and centrifuged before use (15K rpm in a JA20 Beckman rotor, Fullerton, Calif.). The lysate was diluted (to 30% final concentration) with a buffer that contained Ac-DEVD-pNA (250 μM final), dATP (100 μM final) (2′-deoxyadenosine-5′-triphosphate, D6500, Sigma), DTT (2 mM final) (Dithiothreitol, D5545, Sigma); 50 μl of this material were immediately added to plates that contained 12 compounds per well (dried, 20 μM final per compound), and an initial absorbance was read at 390 nm (SpectroMax 250, Molecular Device, Sunnyvale, Calif.). The plates were allowed to incubate for three to four hours. When 90% of the Ac-DEVD-pNA (Bachem L1945) was converted by the activated capsase-3 in the control plate, the screening plates were read again at 390 nm. The change in absorbency was scaled to the fully-activated control (cytochrome c) and the negative control (no compound). Wells that exhibited greater than 5% activation were investigated further in the same assay to elucidate the active compound.

One hundred eighty four thousand compounds were screened from the Abbott Laboratories (Abbott Park, Ill.) screening library in this manner. Of these, twenty-eight compounds were identified as having some stimulating effect in the assay. Of these, six had measurable EC₅₀'s, with PETCM being the most active compound.

### EXAMPLE II

### Preparation of Q-ft, Q30 and Q100

Ten ml of HeLa S-100 (˜60 mg total protein) was loaded on a 1-ml HiTrap Q column (Pharmacia) pre-equilibrated with Buffer A (i.e., 20 mM Hepes-KOH, pH7.5, 10 mM KCl, 1.5 mM Mg Cl2, 1 mM sodium EDTA and 1 mM sodium EGTA, 1 mM dithiothreitol, and 0.1 mM PMSF). The flowthrough (Q-ft) was collected. After being washed with 10-ml of Buffer A, the column was eluted with 10-ml of Buffer A containing 300 mM NaCl, and the eluted protein peak (˜4 l) was collected (Q30). Subsequently, the column was eluted with Buffer A containing 1 M NaCl, and the protein peak (−3 ml) was collected and dialyzed for overnight (Q100).

### EXAMPLE III

### Purification and Identification of PHAP from HeLa S100 Cells

All purification steps were carried out at 4° C., and chromatography was performed on a Pharmacia FPLC system. HeLa cell S-100 was prepared in Buffer A (20 mM Hepes, pH 7.5, 10 mM KCl, 1.5 mM MgCl2, 1 mM EDTA, 1 mM EGTA, 1 mM DTT, and 0.1 mM PMSF) containing protease inhibitors as described (Liu et al., 1996). About 150 ml of HeLa S-100 (˜1 g total protein) was obtained from 25 liters of cell culture. The HeLa S-100 was applied to a Q-Sepharose column (40-ml bed volume) (Pharmacia, Piscataway, N.J.) equilibrated with Buffer A. After washing the column with 250 ml of Buffer A containing 0.3 M NaCl, the stimulatory factor was eluted with Buffer A containing 1 M NaCl and the eluted protein peak was collected (100 ml, ˜125 mg total protein). After adjusting NaCl concentration to 4 M by dissolving NaCl powder, it was loaded on a phenyl-Sepharose column (40-ml bed volume) (manufacturer, city, state) equilibrated with Buffer A containing 4 M NaCl. The activity flew through the column (˜6 mg total protein). After adjusting (NH4)₂SO₄ concentration to 60% saturation, it was applied to a 1-ml phenyl-Sepharose column equilibrated with Buffer A containing 60% saturated (NH4)₂SO₄, and the activity was eluted with a gradient from 60% to 20% saturated (NH4)₂SO₄ in 40 ml of Buffer A. The activity was combined (˜0.7 mg total protein), concentrated to 0.5 ml, and subsequently resolved by a 25-ml Superdex 200 gel filtration column (Pharmacia, Piscataway, N.J.) with Buffer A containing 50 mM NaCl. The active fractions were combined (˜0.45 mg total protein), and finally resolved by a Mono Q 5/5 column with a 300-600 mM NaCl gradient in 40 ml of Buffer A. The activity was eluted at about 500 mM NaCl. The purified proteins were identified as PHAPI and related proteins by Mass-Mass spectrum analysis at Cell Signaling Alliance Facility at UT Southwestern Medical Center (Dallas, Tex.) according to standard procedures.

### EXAMPLE IV

### Purification and Identification of ProT from HeLa S-100 Cells

All purification steps were carried out at 4° C., and chromatography was performed on a Pharmacia FPLC system. One hundred liters of HeLa cell culture were used to obtain 600 ml of Hela S100 (˜3.6 g total protein). Ammonium sulfate concentration was adjusted to 70% saturation, and the precipitated protein was removed by centrifugation. The supernatant (˜0.6 g total protein) was loaded on a phenyl Sepharose column (40-ml bed volume) equilibrated with Buffer A containing 70% saturated (NH4)₂SO₄. After washing the column with 250 ml of Buffer A containing 70% saturated (NH4)₂SO₄, the inhibitory activity was eluted with Buffer A containing 30% saturated (NH4)₂SO₄, and the eluted protein peak was collected (100 ml, ˜60 mg total protein). The activity was dialyzed against Buffer A for overnight and loaded on a 8 ml Mono-Q equilibrated with Buffer A, and subsequently eluted with a gradient of 300-600 mM NaCl in 100 ml of Buffer A. The active fractions were combined (˜1.2 mg total protein), and loaded on a 2-ml hydroxyapatite column equilibrated with Buffer A. A gradient of 0-100 mM KPO₄ (pH 7.5) in 20 ml of Buffer A was performed to elute the inhibitory factor. The active fractions were combined (˜0.4 mg protein), concentrated to 1 ml, and subjected to 2 runs of gel filtration on a Superdex 200 column (Pharmacia, Piscataway, N.J.) eluted with Buffer A. The active fractions were combined (˜0.2 mg), and resolved by a 1-ml Mono-Q column with a gradient of 300-600 mM NaCl in 30 ml of Buffer A. The purified protein was identified as prothymosin-alpha by Mass-Mass spectrum analysis at Cell Signaling Alliance Facility at UT Southwestern Medical Center (Dallas, Tex.) according to standard procedures.

### EXAMPLE V

### Cloning of PHAPI and ProT, and Production of Recombinant Proteins

PHAPI open reading frame (ORF) was amplified by PCR from image clone AA488559 (Incyte Genomics Inc., Palo Alto, Calif.) using primers CGGCAGATCTCTGGATCCATGGAGATGGGCAGACGGATTC (SEQ ID NO:1) and CGCCGTCGACTTAGTCATCATCTTCTCCCTC (SEQ ID NO:2). The amplified product was subcloned into BamHI/SalI sites of pET-28a(+) vector (Novagen, Milwaukee, Wis.). The plasmid was used to express recombinant His-tagged PHAPI in BL21 (DE3) strain and the protein was purified using NTA-agarose (Qiagen, Valencia, Calif.) followed by Q-Sepharose chromatography. ProT ORF was amplified by PCR from image clone B315161 (Incyte) using primers CCGGCATATGTCAGACGCAGCCGTAGAC (SEQ ID NO:3) and CCGGCTCGAGGTCATCCTCGTCGGTCTTCTG (SEQ ID NO:4). The amplified product was subcloned into NdeI/XhoI sites of pET-21b vector (Novagen). The plasmid was used to express recombinant His-tagged ProT in BL21 (DE3) strain, and the protein was purified using NTA-agarose (Qiagen, Valencia, Calif.) followed by Q-Sepharose chromatography.

### EXAMPLE VI

### RNAi of ProT and Cell Death Analysis of Hela Cells

Double-strand siRNA UCACCACCAAGGACUUAAA (SEQ ID NO:5), corresponding to a region of ProT mRNA, with dTdT overhead in 3′-ends, was synthesized by Dharmacon (Lafayette, Colo.) to disrupt ProT mRNA in Hela cells. Double-stranded siRNA GCAGCACGACUUCUUCAAGU (SEQ ID NO:6) (3′-end dTdT overheads) corresponding to a region of green fluorescence protein (GFP) was used as the negative control. DNA primers ATGATCTCGGATGACCAAAC (SEQ ID NO:7) and GGAGGCGGCTGCGGCGAGCA (SEQ ID NO:8) were used for RT-PCR of ProT. DNA primers TCCACCACCCTGTTGCTGTA (SEQ ID NO:9) and ACCACAGTCCATGCCATCAC (SEQ ID NO:10) were used for RT-PCR of GAPDH. HeLa cells were grown in 6-well plates. Transfection of dsRNA to HeLa cells was performed using OligofectAmine reagent (Invitrogen, Carlsbad, Calif.) according to standard procedure. The final siRNA concentration of the transfection was 16 nM. Two days after transfection, RT-PCR was performed to measure ProT mRNA level, cells were treated with 10 mJ/cm² of UV light using UV Stratalinker 1800 (Stratagene, La Jolla, Calif.), and cell death was accessed at an indicated time. Dead cells were stained by Hoechst 33342 (Sigma, St. Louis, Mo.) and counted under microscope. For caspase-3 activity measurement, cells were harvested with or without UV treatment as indicated, and lysed in Buffer A containing protease inhibitors by three cycles of freeze-and-thaw, the measurement was performed in a 100-μl system containing 10 μM DEVD fluorogenic substrate (CalBiochem, La Jolla, Calif.) and 20 μg cytosolic protein at 30° C. using a Xfluor4 spectrometry reader (TECAN Austria).

1. A compound comprising alpha-(trichloromethyl-4-Pyridineethanol) (PETCM) and derivatives thereof.

2. The compound of claim 1, wherein said compound is alpha-(trichloromethyl-4-Pyridineethanol) (PETCM).

3. The compound of claim 1, wherein said compound is isolated by high throughput screening.

4. A method of identifying at least one protein which inhibits or activates an apoptopic pathway comprising the steps of:
a) preparing fractions of a cellular extract;
b) exposing said fractions to PETCM and determining whether apoptosis activation or inhibition occurs in each of said fractions;
c) purifying said fractions which exhibit apoptosis activation or inhibition upon exposure to PETCM; and
d) identifying from said purified fractions at least one protein, wherein said at least one protein inhibits or activates apoptosis in said apoptopic pathway.

5. The method of claim 4 wherein said at least one protein which activates apoptosis is selected from the group consisting of PHAPI, PHAP12a and PHAPIII.

6. The method of claim 4 wherein said at one protein which inhibits apoptosis is promothymosin-alpha.

7. A method of activating a caspase pathway in a cell comprising the step of exposing PETCM to said cell in an amount sufficient to effect said activation.

8. The method of claim 7, wherein said cell is mammalian.

9. The method of claim 8, wherein said mammalian cell is malignant.

10. The method of claim 9, wherein said malignant cell is selected from the group consisting of a colon cancer cell, a prostate cancer cell, a leukemia cell, a melanoma cell, a lymphoma cell, a cervical carcinoma cell and a glioblastoma cell.

11. The method of claim 7, wherein said caspase pathway is the caspace-3 pathway.

12. The method of claim 11, wherein said PETCM is exposed to said cell in a range of approximately 0.1 uM to 1.0 mM.

13. The method of claim 12, wherein said PETCM is exposed to said cell at a concentration of 0.2 mM.

14. A method of inducing apoptosome formation in a cell, wherein said formation is inhibited by ProT, comprising the step of exposing PETCM to said cell in an amount sufficient to effect said induction.

15. A method of inducing function of PHAP protein, in a cell, inhibited by prothymosin-alpha (ProT) comprising the step of exposing PETCM to said cell in an amount sufficient to induce said function.

16. A method of reversing inhibition of caspace-3 activation, in a cell, wherein said inhibition is induced by ProT, comprising the step of exposing PETCM to said cell in an amount sufficient to effect said reversal.

17. A method of negatively regulating caspase-9 activation in a cell comprising the step of exposing ProT to said cell in an amount sufficient to negatively regulate said activation.

18. A method of promoting caspase activation in a cell, subsequent to apoptosome formation, comprising administering PHAP protein to said cell in an amount sufficient to effect said activation.

19. A method of identifying regulators of apoptosome formation comprising the steps of:
a) preparing extracts of mammalian, malignant cells;
b) exposing said extracts to a probe, wherein said probe comprises a nucleotide sequence encoding prothymosin-alpha, for a time and under conditions sufficient for complexes to form between said probe and nucleic acid sequences in said extracts; and
c) detecting complex formation between said probe and said nucleic acid sequences in said extracts, wherein said nucleic acid sequences encode regulators of apoptosome formation.
